(19) United States
(12) Patent Application Publication
(10) Pub. No.: US 2006/0128006 A1
(43) Pub. Date: Jun. 15, 2006

(54) Hydrodynamic capture and release mechanisms for particle manipulation

(76) Inventors: Antimony L. Gerhardt, Springfield, LA (US); Gwendolyn L. Gerhardt, Springfield, LA (US); Rebecca Braff Maxwell, Newton, NJ (US); Joel Voldman, Somerville, MA (US); Martha Gray, Arlington, MA (US); Martin Schmidt, Reading, MA (US); Mehmet Toner, Wellesley, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 11/146,581

(22) Filed: Jun. 7, 2005

Related U.S. Application Data
(63) Continuation-in-part of application No. 10/778,831, filed on Feb. 13, 2004.
Application No. 10/778,831 is a continuation of application No. 09/710,032, filed on Nov. 10, 2000, now Pat. No. 6,692,952.
(60) Provisional application No. 60/164,643, filed on Nov. 10, 1999.

(51) Int. Cl. H01L 21/00; C12M 1/34

(52) U.S. Cl. 435/287.1; 438/1

(57) ABSTRACT

A cell analysis and sorting apparatus is capable of monitoring over time the behavior of each cell in a large population of cells. The cell analysis and sorting apparatus contains individually addressable cell locations. Each location is capable of capturing and holding a specified number of cells, and selectively releasing that specified number of cells from that particular location. In one aspect of the invention, the cells are captured and held in wells, and released using vapor bubbles as a means of cell actuation. Disclosed are: a cell manipulation apparatus design; various resistive heater configurations for nucleating microbubbles; various well designs, each in communication with a nucleation chamber or channel, for capturing a specified number of cells; and methods of fabrication and cell population manipulation.

### CROSS-REFERENCE TO RELATED APPLICATIONS

This application is a continuation-in-part of U.S. patent application Ser. No. 10/778,831, filed on Feb. 13, 2004, which is a continuation of U.S. patent application Ser. No. 09/710,032, filed on Nov. 10, 2000, now U.S. Pat. No. 6,692,952, which claims priority to U.S. Provisional Application No. 60/164,643, filed on Nov. 10, 1999, each of which is incorporated herein in their entirety by reference.

### FIELD OF THE INVENTION

This invention relates to particulate analysis and sorting devices and methods for manipulating particulates including, for example, living cells. More particularly, the invention relates to particulate analytical and sorting systems that can capture and hold individual particulates or set numbers of particulates at known locations and then selectively release certain of these particulates. Methods of manipulating the particulates via microfluidic control are also disclosed.

### BACKGROUND OF THE INVENTION

Many recent technological advances have enhanced the study of cellular biology and biomechanical engineering, most notably by improving methods and devices for carrying out cellular analysis. For example, in the past decade an explosion in the number of optical probes available for cell analysis has enabled an increase in the amount of information gleaned from microscopic and flow cytometric assays. Microscopic assays allow the researcher to monitor the time-response of a limited number of cells using optical probes. Flow cytometry, on the other hand, uses optical probes for assays on statistically significant quantities of cells for sorting into subpopulations.

However, these mechanisms alone are often insufficient for time-dependent analysis. Microscopic assays can only track a few cells over time and do not allow the user to track the location of individual cells. With flow cytometry, the user can only observe each cell once and can only easily sort a cell population into three subpopulations. Flow cytometry techniques fail to provide for analysis of the same cell multiple times, or for arbitrary sorting of subpopulations. These kinds of bulk assay techniques produce mean statistics, but cannot provide the researcher with distribution statistics.

Advances in microsystems technology have also influenced many applications in the fields of cell biology and biomedical engineering. Scaling down to the micron level allows the use of smaller sample sizes than those used in conventional techniques. Additionally, the smaller size and ability to make large arrays of devices enables multiple processes to be run in parallel.

Integrated circuits have been fabricated on silicon chips since the 1950s, and as processing techniques improve, the size of transistors continues to shrink. The ability to produce large numbers of complex devices on a single chip sparked interest in fabricating mechanical structures on silicon as well. The range of applications for micro electromechanical systems (MEMS) is enormous. Accelerometers, pressure sensors, and actuators are just a few of the many MEMS devices currently produced. For example, in 2003, P. Deng et al. in Design and characterization of a micro single bubble actuator, Proc. 12^{th} International Conference on Solid State Sensors, Actuators, and Microsystems (Transducers '03), vol. 1, Boston, Mass., 2003, p. 647-650, which is hereby incorporated by reference, described using a single bubble actuator for actions such as mixing in micro-bio-analytical systems. Another application of MEMS is in biology and medicine. Micromachined devices have been made for use in drug-delivery, DNA analysis, diagnostics, and detection of cell properties.

Manipulation of cells is another application of MEMS. For example, in the early 1990's, Sato et al. described in his paper, which is hereby incorporated by reference, Individual and Mass Operation of Biological Cells using Micromechanical Silicon Devices, Sensors and Actuators, 1990, A21-A23: 948-953, the use of pressure differentials to hold cells. Sato et al. microfabricated hydraulic capture chambers that were used to capture plant cells for use in cell fusion experiments. Pressure differentials were applied so that single cells were sucked down to plug an array of holes. Cells could not be individually released from the array, however, because the pressure differential was applied over the whole array, not to individual holes.

Bousse et al. in his paper, which is hereby incorporated by reference, Micromachined Multichannel Systems for the Measurement of Cellular Metabolism, Sensors and Actuators B, 1994, 20:145-150, described arrays of wells etched into silicon to passively capture cells by gravitational settling. Multiple cells were allowed to settle into each of an array of wells where they were held against flow due to the hydrodynamics resulting from the geometry of the wells. Changes in the pH of the medium surrounding the cells were monitored by sensors in the bottom of the wells, but the wells lacked a cell-release mechanism, and multiple cells were trapped in each well. Another known method of cell capture is dielectrophoresis (DEP). DEP refers to the action of neutral particles in non-uniform electric fields. Neutral polarizable particles experience a force in non-uniform electric fields that propels them toward the electric field maxima or minima, depending on whether the particle is more or less polarizable than the medium it is in. By arranging the electrodes properly, an electric field may be produced to stably trap dielectric particles.

Microfabrication has been utilized to make electrode arrays for cell manipulation since the late 1980s. Researchers have successfully trapped many different cell types, including mammalian cells, yeast cells, plant cells, and polymeric particles. Much work involves manipulating cells by exploiting differences in the dielectric properties of varying cell types to evoke separations, such as separation of viable from non-viable yeast, and enrichment of CD34+ stem cells from bone marrow and peripheral blood stem cells. More relevant work on trapping cells in various two- and three-dimensional microfabricated electrode geometries has been shown by several groups. However, trapping arrays of cells with the intention of releasing selected subpopulations of cells has not yet been widely explored. Additionally, DEP can potentially induce large temperature changes, causing not only convection effects but also profoundly affecting cell physiology.

These studies demonstrate that it is possible to trap individual and small numbers of cells in an array on a chip, but without the ability to subsequently manipulate and selectively release individual cells. This inability to select or sort based on a biochemical measurement poses a limitation to the kinds of scientific inquiry that may be of interest.

The currently available mechanisms for carrying out cell analysis and sorting are thus limited in their applications. There is thus a need for an improved method and apparatus for sorting and releasing large quantities of cells that can easily and efficiently be used. In addition, there is a need for an analysis and sorting device that allows the user to look at each cell multiple times, and to track many cells over time. Finally, there is a need for a cell sorter that lets the user know the cell locations and be able to hold and selectively release the cells so that the user can arbitrarily sort based on any aspect of the cells' characteristic during time-responsive assays.

### SUMMARY OF THE INVENTION

The present invention provides a particulate sorting apparatus that is capable of monitoring over time the behavior of each particulate in a large population of particulates. The particulate analysis and sorting apparatus contains individually addressable particulate locations. Each location is capable of capturing and holding a single particulate, and selectively releasing that particulate from that particular location. Alternatively, each location can be designed to selectively capture, hold, and then release multiple particulates. In one aspect of the invention, the particulates are captured and held in wells, and released using vapor bubbles as a means of particulate election. In another aspect of the invention, the particulates are captured, held and released using electric field traps. The invention is particularly useful in sorting cells and other biological matters. It should be understood that the terms “cell,” “particle,” and “particulate” are used in various locations herein but, unless otherwise indicated, the term is intended to encompass generally. For example, the “cell” could be a bead, lymphocyte, bacteria, cellular fragment, viral particle, fungi, particle, biological molecule, ions, or nanoparticle.

Applications for the invention may include but are not limited to: investigating temporal cell response to various stimuli; phenotype inhomogeneities in a nominally homogeneous cell population; molecular interactions such as receptor-ligand binding or protein-protein interactions; signal transduction pathways such as those involving intracellular calcium; gene expression such as with immediate-early genes either in response to environmental stimuli or for cell-cycle analysis; and heterogeneity in gene expression to investigate stochastic processes in cell regulation. Other opportunities for use of the invention may include but are not limited to: drug discovery, such as in report gene based assays; fundamental biological issue assays, such as dealing with kinetics of drug interactions with cells and sorting based on interesting pharmocodynamic responses; and clinical setting applications such as to diagnose disease, monitor progression, and monitor treatment by looking for abnormal time responses in patients' cells.

According to one aspect of the present invention, the particulate analysis and sorting apparatus has an array of geometric sites for capturing particulates traveling along a fluid flow. The geometric sites are arranged in a defined pattern across a substrate such that individual sites are known and identifiable. Each geometric site is configured and dimensioned to hold a single particulate. Additionally, each site contains a release mechanism to selectively release the single particulate from that site. Because each site is able to hold only one particulate, and each site has a unique address, the apparatus allows the user to know the location of any particular particulate that has been captured. Further, each site is independently controllable so that the user is able to arbitrarily capture particulates at select locations, and to release particulates at various locations across the array.

In one embodiment of the present invention, the particulates are biological cells and the geometric sites are configured as wells. As a fluid of cells is flown across the array of specifically sized wells, cells will fall into or be drawn into the wells and become trapped. Each well is sized and shaped to capture only a single cell, and is configured such that the cell will not escape into the laminar flow of the fluid above the well.

The single cell or other particulate can be held inside the well by gravitational forces. Alternatively, the particulate can be held in the well by a pressure gradient. A particulate can be captured in the well by a pressure differential between the fluid in which the particulates are flowing and the fluid in a chamber or another stream of fluid fluidically connected to the capture site. By controlling the flow rates between the two fluid flows, the pressure drop that is created can capture a particulate.

In another embodiment of the present invention, a three-dimensional electric field trap can form the geometric sites. Each trap can comprise four electrodes arranged in a trapezoidal configuration, where each electrode represents a corner of the trapezoid. The electric fields of the electrodes create a potential energy well for capturing a single cell or other particulate within the center of the trap. By removing the potential energy well of the trap, the cell is ejected out of the site and into the fluid flow around the trap. Microfluidic actuation can be used in conjunction with electronic control or as alternative release mechanism, as described below. Ejected cells can then be entrained in a fluid flow and collected or discarded.

In one preferred embodiment of the invention, each well or capture site can further be attached via a narrow channel to a chamber located below (or otherwise adjacent) the well. The term chamber as used herein is intended to include not only closed spaces, e.g., surrounded by four walls or one cylindrical wall, but more generally encompass any space adjacent to the capture site where microfluidic actuation can occur, e.g., a channel or additional stream of fluid. Microfluidic actuation is used to release individual captured cells. Within the chamber is a heating element that is able to induce bubble nucleation, the mechanism for releasing the cell from the site. The heating element can be a planar resistive heating element, comprising a resistor with a narrowed portion forming the bubble nucleation site at which a bubble is formed. The planar resistive heating element forms a surface of the chamber. The bubble creates volume expansion inside the chamber which, when filled with fluid, will displace a jet of fluid out of the narrow channel and eject the particulate out of the well. Bulk fluid flow will sweep the ejected particulate away to be either collected or discarded.

In another aspect of the invention, integrated systems are proposed. The system can be a microfabrication-based dynamic array cytometer (μDAC) having as one of its components the cell analysis and sorting apparatus previously described. To analyze a population of cells, the cells can be placed on a cell array chip containing a plurality of cell sites. The cells are held in place within the plurality of cell sites in a manner similar to that described above. Different mediums, concentrations, or stimuli, for example, may be introduced along the columns of the cell sites. The cells can be analyzed, for example, by photometric assay. Using an optical system to detect fluorescence, the response of the cells can be measured, with the intensity of the fluorescence reflecting the intensity of the cellular response. Once the experiment is complete, the cells exhibiting the desired response, or intensity, may be selectively released into a cell sorter to be further studied or otherwise selectively processed. Such an integrated system would allow researchers to also look at the cell's time response.

Further features and advantages of the present invention as well as the structure and operation of various embodiments of the present invention are described in detail below with reference to the accompanying drawings.

### BRIEF DESCRIPTION OF THE DRAWINGS

This invention is pointed out with particularity in the appended claims. The above and further advantages of this invention may be better understood by referring to the following description when taken in conjunction with the accompanying drawings, in which:

FIG. 1A is a cross-section schematic diagram of one embodiment of the present invention, illustrating a gravity-based capture mechanism;

FIG. 1B is a cross-section schematic diagram of another embodiment of the present invention, illustrating a fluid pressure gradient capture mechanism;

FIG. 1C is a top-view schematic diagram of a monolithic or planar embodiment of the present invention shown in FIG. 1B with a fluid pressure gradient capture mechanism;

FIG. 1D is a top-view cross-section schematic diagram of another monolithic or planar embodiment of the present invention shown in FIG. 1B;

FIG. 1E is a top-view cross-section schematic diagram of another monolithic or planar embodiment of the present invention shown in FIG. 1B;

FIGS. 2A, 2B and 2C are schematic diagrams of yet another embodiment of the present invention, illustrating a electric field capture mechanism;

FIGS. 3A and 3B show a top-down view of the cell sorting apparatus of FIG. 2A;

FIGS. 4A, 4B, 4C, 4D, and 4E are schematic diagrams of a microfluidic actuator in operation according to the present invention;

FIG. 5 is a schematic illustration of another aspect of the present invention in which a particulate or cell sorting apparatus is integrated into a fluorescence-detecting system;

FIGS. 6A and 6B are schematic diagrams of fluid flow paths in a cell capture and sorting apparatus according to the invention;

FIGS. 7A and 7B are schematic diagrams further illustrating microbubble formation in a cell capture and sorting apparatus according to the invention;

FIGS. 8A, 8B and 8C are schematic illustrations of an in-plane resistive heating element for use in a microfluidic actuator according to the invention;

FIGS. 9A, 9B and 9C are schematic illustrations of an out-of-plane resistive heating element for use in a microfluidic actuator according to the invention;

FIGS. 10A, 10B and 10C are schematic illustrations of a thin-plane resistive heating element for use in a microfluidic actuator according to the invention;

FIG. 11 is a schematic flow for fabricating an out-of-plane resistive heating element;

FIG. 12 is a schematic flow for fabricating an in-plane resistive heating element;

FIG. 13 is a schematic flow for fabricating a thin-plane resistive heating element;

FIG. 14A is a diagram showing an exemplary system input pattern as a function of time vs. voltage.

FIG. 14B is a diagram of a microbubble for determining the diameter and eccentricity of the microbubble.

FIG. 14C is a diagram of a microbubble for determining the centricity of the microbubble.

FIG. 15 is a graph of time vs. average diameter of a microbubble showing an exemplary system response to a single pulse of voltage applied to an in-plane resistive heating element;

FIG. 16A is a graph of time vs. average diameter of a microbubble, showing typical complete system responses to a single pulse of voltage applied to an out-of-plane actuator at time t=0 s;

FIG. 16B is a graph of time vs. average diameter of a microbubble, showing typical complete system responses to a single pulse of voltage applied to an in-plane actuator at time t=0 s;

FIG. 17 is a graph of time vs. average diameter of a microbubble showing the system response to a single pulse of voltage applied to a low-resistance, in-plane resistive heating element;

FIG. 18 shows a graph of eccentricity and centricity, quantification of shape, for an out-of-plane and an in-plane resistive heating element; and

FIG. 19 shows graphs of applied pulse width vs. slow transient dissipation time for a microbubble, applied pulse width vs. average microbubble diameter, and slow transient dissipation time for a microbubble vs. the average microbubble diameter.

### DETAILED DESCRIPTION OF THE INVENTION

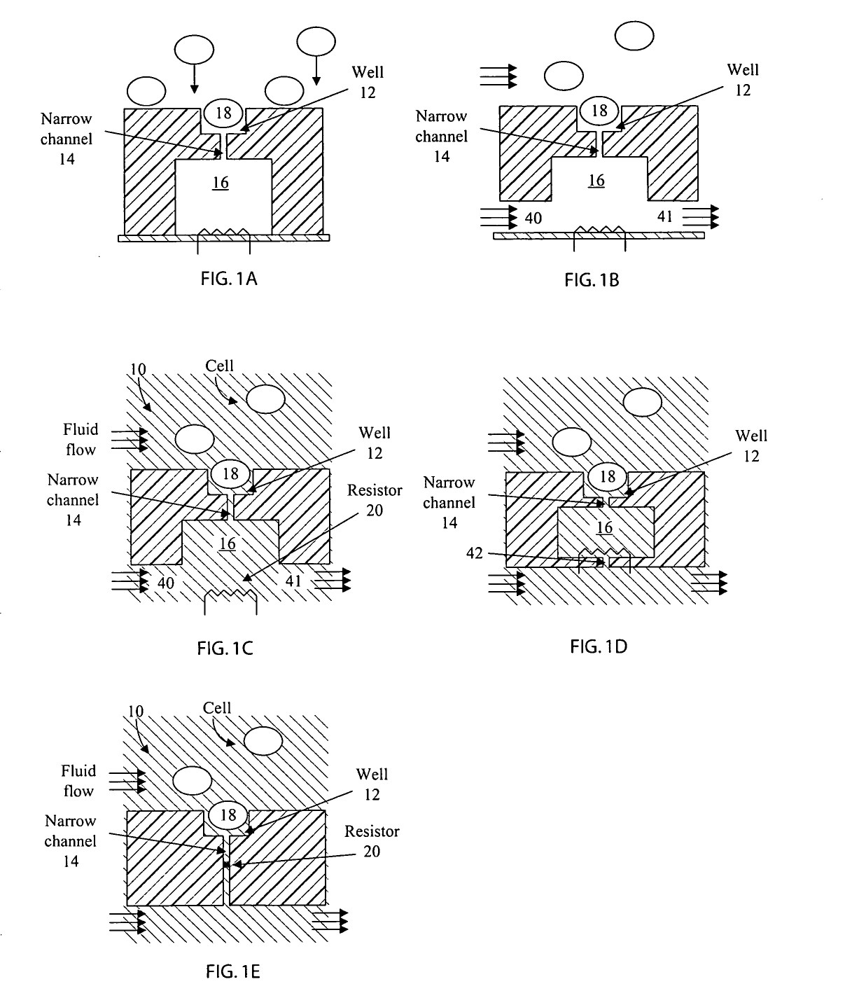

FIGS. 1A-1E illustrate exemplary capture mechanisms according to the present invention. In FIG. 1A, a particulate site 10, shown in cross-section, contains a well 12 that is sized and shaped to hold a single particulate 18. Connected to the bottom of the well 12 is a narrow channel 14 that opens into a chamber 16 situated below the well. In this particular example, the well 12 and narrow channel 14 are etched out of a silicon wafer or casted from a material such as polydimethylsiloxane (PDMS). The silicon wafer or cast is attached to a glass slide on which there is a heater 20, and the alignment is such that the heater 20 is sealed inside the chamber 16, which is filled with a fluid such as water or cellular medium.

The well 12 functions as a capture and hold mechanism to trap a single particulate. In the embodiment of FIG. 1 A, gravity is utilized as the capture mechanism to trap the particulate in well 12. In operation, fluid containing particulates are flown over the top of the apparatus, and then the flow is stopped. As shown in FIG. 1A, the particulates then settle and gravitational forces will allow one particulate 18 to fall into and become trapped within the well 12. At this point the flow is started again, and the cell in the well is trapped while the cells not in wells are flushed away by convection. The well 12 is dimensioned and configured to hold only one cell 18 within the well 12 at a time or to hold a chosen number of cells. In addition, the well 12 is configured such that the cell 18 will not be swept out of the well due to laminar or fluid flow above.

In another embodiment of the invention, shown in FIG. 1B, a pressure gradient is utilized as the capture mechanism to trap a cell in well 12. This is achieved using a pressure differential between a fluid in chamber 16 and the fluid flow of cells over the cell sites. By controlling the flow rates of the two fluid flows, a pressure drop is created that will trap a particulate in well 12. The cell is held in well 12 due to the pressure gradient and the geometry of well 12.

FIGS. 1C, 1D, and 1E show planar embodiments of the invention depicted in FIG. 1B. Instead of a vertical alignment of the well, narrow channel, and chamber (as in FIGS. 1A and 1B), the components in FIGS. 1C, 1D, and 1E are arranged in a planar manner.

FIG. 1C, shown in top-view, is a planar embodiment of the invention in FIG. 1B, shown in top-view.

FIG. 1D, shown in top-view, is another planar embodiment of the invention in FIG. 1B. In this embodiment of the invention, heating element 20 is located in chamber 16.

FIG. 1E, shown in top-view, contains a well 12 to hold a particulate 18. A heating element 20 is located within a narrow channel 14, which connects well 12 to one of the fluid flows used to achieve the pressure differential to capture a cell 18 in well 12.

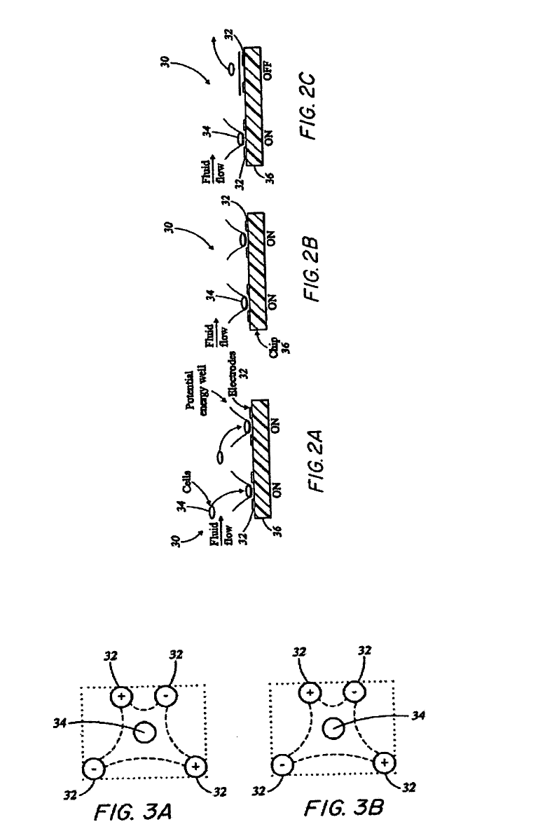

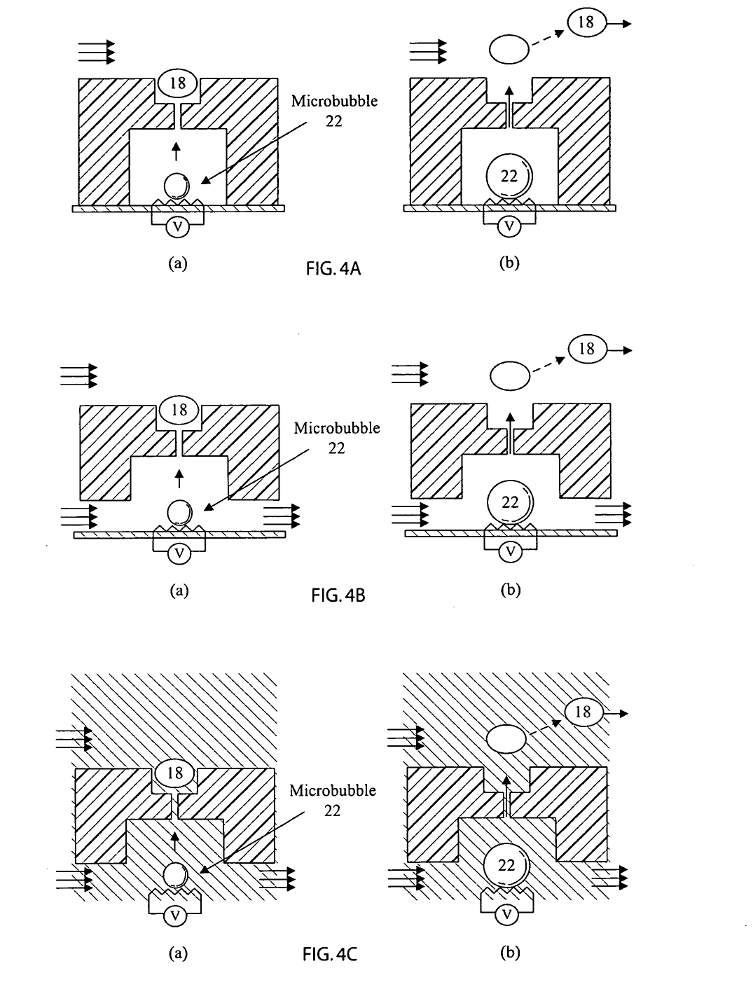

In another exemplary capture mechanism, the cell site 30 can include electric field traps. FIGS. 2A-2C show, in cross-section, two cell sites on a substrate such as a microfabricated chip 36. Each site includes a plurality of electrodes 32. Preferably, each cell site 30 contains four electrodes, positioned in a trapezoidal configuration, as seen in FIGS. 3A and 3B. The cell site 30 is configured and positioned such that only one cell can be held within the site. The electrodes 32 create a non-uniform electric field trap within which a single cell 34 can be held and subsequently released.

In the electric field embodiment, cells in fluid medium flow over the cell sites 30, as shown in FIG. 2A. By adjusting the electric field of each electrode 32, a potential energy well can be created within each cell site 30. The potential energy well is of sufficient strength to capture a single cell 34 traveling along the fluid flow and to hold the cell 34 within the center of the trap, as seen in FIG. 2B. When the operator elects to release a cell 34, the electric fields of the electrodes 32 forming the trap are adjusted to initiate release. FIG. 2C shows how this in turn removes the potential energy well, releasing the cell 34 back into the fluid flow. The cell 34 can then be collected or discarded.

The electrodes forming the electric field trap can be thin-film poles formed of gold. This creates a three-dimensional electric field trap that is effective in holding a cell against the laminar flow of the fluid surrounding the electrodes. Further, while only one or two cell sites are illustrated, it is understood that the drawings are merely exemplary of the kind of site that can be included in the cell sorting apparatus of the present invention. The cell sorting apparatus can contain anywhere from a single cell site to an infinite number of cell sites, for sorting mass quantities of cells. Moreover, while the embodiments herein are described as holding cells, it is understood that what is meant by cells includes but is not limited to beads, lymphocytes, bacteria, cellular fragments, viral particles, fungi, particles, biological molecules, ions, or nanoparticles.

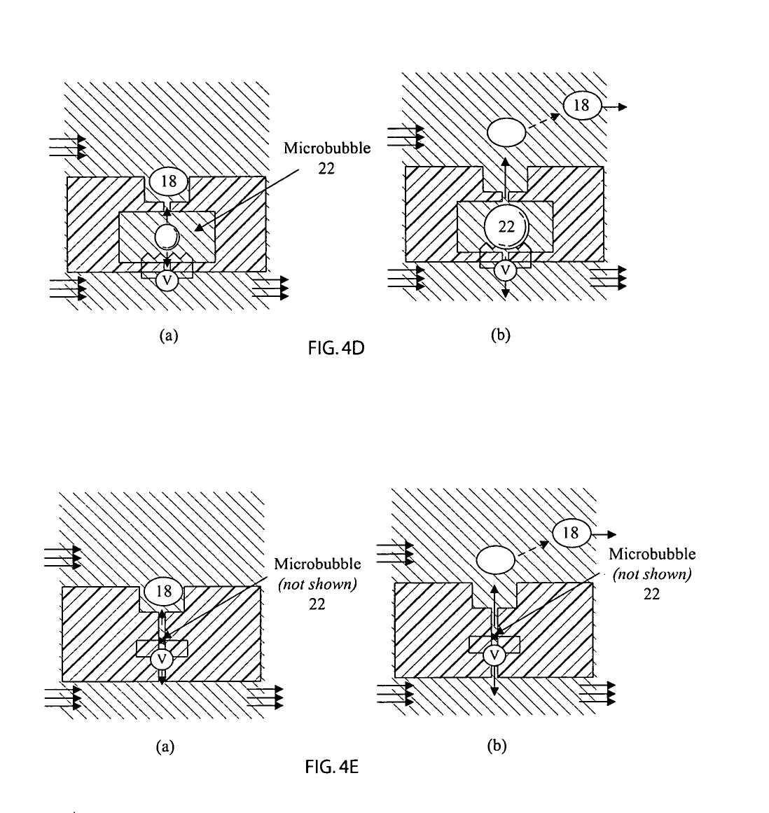

FIGS. 4A-4E illustrate the basic release mechanism of the present invention. When it is desired to release cell 18 from the well 12, the operator can apply a current pulse to the heating element 20. The heating element 20 is then heated to a temperature to initiate vapor bubble nucleation at the surface of the heating element 20, as seen in FIG. 4A. In FIG. 4B, a microbubble 22 is formed inside the chamber 16, creating a volume displacement. By adjusting the voltage, current, and duration of the pulse applied to the heating element 20, the operator can control the size of the microbubble 22. When the microbubble 22 is of sufficient size, the volume expansion in the chamber will displace a jet of fluid out of the narrow channel 14, ejecting the cell 18 out of the well 12. The released cell 18 can be swept into the bulk fluid flow outside the well 12, to be later collected or discarded.

FIGS. 4C, 4D, and 4E depict the release mechanisms used in the planar embodiments of the invention (as shown in FIGS. 1C, 1D, and 1E). FIG. 4C uses the same release mechanism as shown in FIG. 4B, with the device aligned in a planar manner. FIG. 4D uses the same release mechanism as shown in FIG. 4B, with the heating element being located along a surface of the chamber. FIG. 4E uses the same release mechanism shown in FIG. 4B, with the heating element being located within the narrow channel.

In one embodiment of the invention, the particulates are cells. Experiments may be performed on the trapped cells, such as by adding a reagent across the entire population or by using laminar flow or geometry to expose columns or groups of cells to different reagents. When the experiments are concluded, the cells exhibiting the desired characteristics may be selectively released from the wells. Because the cell sorting apparatus of the present invention allows the operator to know the location of each cell in the array of cell sites, the operator is able to manipulate the cells and arbitrarily sort the cells based on their characteristic under time-responsive assays. One such method can employ scanning techniques to observe dynamic responses from cells.

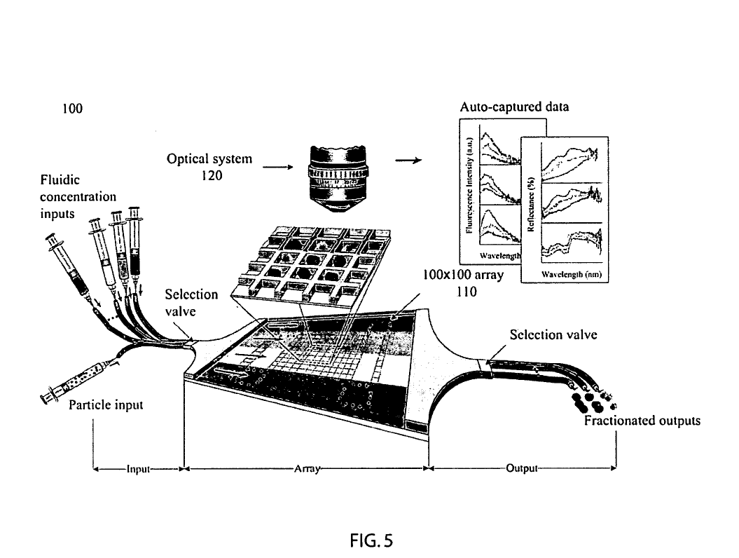

As shown in FIG. 5, an integrated cellular analysis system 100 is proposed in which cells are tested using light-emitting assays to determine the cell's response to stimuli over time. The integrated system can be a microfabrication-based dynamic array cytometer (μDAC). Cells undergoing analysis can be placed on a cell array chip 110 similar to the cell sorting apparatus above, to be held in place within the plurality of cell sites, such as those described above. Using an optical system 120 to detect fluorescence, the response of the cells can be measured, with the intensity of the fluorescence reflecting the intensity of the cellular response. Once the experiment is complete, the cells exhibiting the desired response, or intensity, may be selectively released, to be collected or later discarded. Alternatively, cells exhibiting the desired response can be selectively retained while the others are purged. Such integrated systems allow researchers to look at the cell's time response in response to various stimuli.

Any light-emitting assay in which the cell's response may vary in time is suited for study using this proposed system. It is ideally suited for finding phenotype inhomogeneities in a nominally homogeneous cell population. Such a system could be used to investigate time-based cellular responses for which practical assays do not currently exist. Instead of looking at the presence/absence or intensity of a cell's response to stimulus, the researcher can look at its time response. Furthermore, the researcher can gain information about a statistically significant number of cells without the potential of masking important differences as might occur in a bulk experiment. Specific applications may include the study of molecular interactions such as receptor-ligand binding or protein-protein interactions. Signal transduction pathways, such as those involving intracellular calcium, can also be investigated.

An advantage of the proposed integrated system is that the full time-response of all the cells can be accumulated and then sorting can be performed. This is contrasted with flow cytometry, where each cell is only analyzed at one time-point and sorting must happen concurrently with acquisition. Geneticists can look at gene expression, such as with immediate-early genes, either in response to environmental stimuli or for cell-cycle analysis. Another large application area is drug discovery using reporter-gene based assays. The integrated system can also be used to investigate fundamental biological issues dealing with the kinetics of drug interactions with cells, sorting and analyzing cells that display interesting pharmacodynamic responses. Another application is looking at heterogeneity in gene expression to investigate stochastic processes in cell regulation. Finally, once temporal responses to certain stimuli are determined, the integrated system can be used in a clinical setting to diagnose disease and monitor treatment by looking for abnormal time responses in patients' cells.

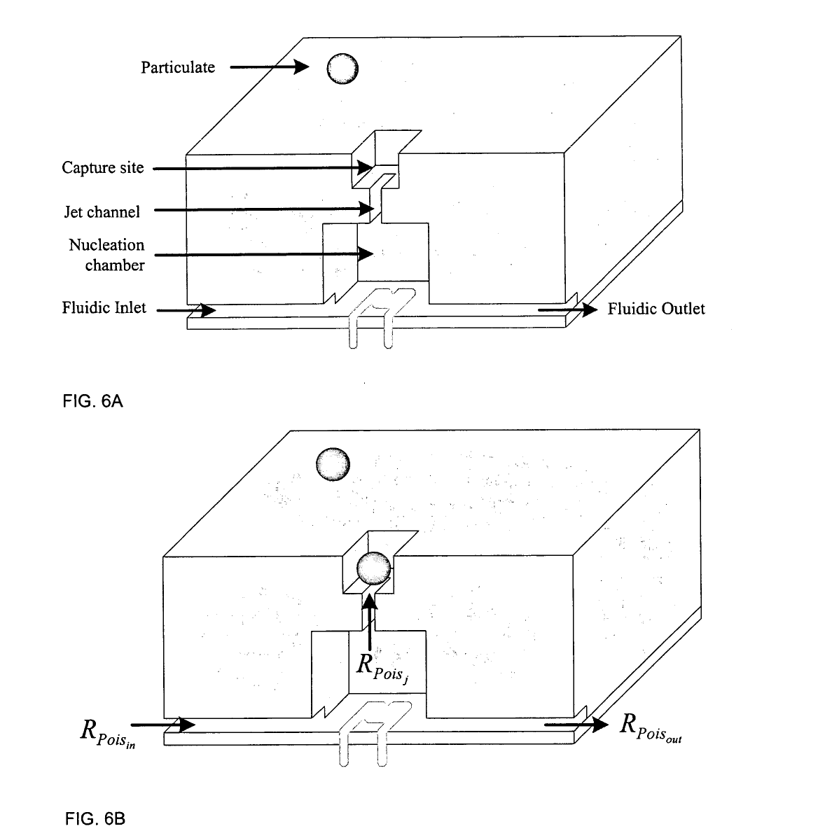

The fluidic system as illustrated in FIGS. 6A and 6B is designed to capture a particulate with a pressure differential between the header in which the particles flow (illustrated at the top of each device schematic) and the nucleation chamber 16 or second fluid flow. By engineering the fluidic resistance in the narrow channel 14 and the fluid inlet and outlet channels, where applicable, and controlling the flow rates in the headers, a pressure drop between the headers will ensure particulate capture at the capture site. The particulate is held in the site against the flow via the pressure gradient and the geometry of the well.

Neglecting gravity, a lumped element model of the Poiseuille flow resistance of a section of channel is defined as

$${R}_{Pois} = \frac{Δ P}{Q} \qquad ( 1 )$$

where ΔP is the pressure gradient between two points along a channel of length L and R_{Pois} is the fluidic resistance of that section of pipe. The pressure drop is related to the flow Q by

$$Δ P = \frac{12 μ L}{{WH}^{3}} Q \qquad ( 2 )$$

where W is the width of the channel and H is the height of the channel. For a circular cross section, the flow rate Q is

$$Q = \frac{π \cdot {r}_{ch}^{4}}{32 μ} K \qquad ( 3 )$$

where r_{ch} is the channel radius, and K is the pressure gradient defined as

$$K = \frac{Δ P}{L} \qquad ( 4 )$$

Solving for R_{Pois} yields

$${R}_{Pois} = \frac{32 μ L}{π \cdot {r}_{ch}^{4}} \qquad ( 5 )$$

For square channels, the hydraulic radius is used for r_{ch} where the hydraulic diameter is

$${D}_{h} \approx \frac{4 \times area}{perimeter} \qquad ( 6 )$$

In the illustrated embodiment of FIGS. 6A and 6B, the capture site can be a cylinder with a diameter of 30 μm and a height of 15 μm (although for ease of illustration it is shown rectangular in the figure). The nucleation chamber can be a rectangular solid with dimensions of 400 μm in length and 300 μm in width and height. The inlet and outlet can be rectangular solids with dimensions of 250 μm in length by 6 μm in width and height.

For particle ejection, the Poiseuille flow parameters are preferably set such that the fluidic resistance of the narrow channel 14 is substantially less than the inlet and outlet channels to the nucleation chamber. The header in which the particles flow (illustrated at the top of each device schematic) and the nucleation chamber 16 or second fluid flow header have the least resistance. Meaning,
R_{Pois}_{ⱼ}<<R_{Pois}_{ᵢₙ}≈R_{Pois}_{ₒᵤₜ}<<R_{Pois}_{_{header}}≈R_{Pois}_{_{chamber}} (7)
where the subscript j denotes the narrow channel, in denotes the inlet channel, out denotes the outlet channel, header denotes the header in which the particles flow or the second fluid flow header, and chamber denotes the nucleation chamber.

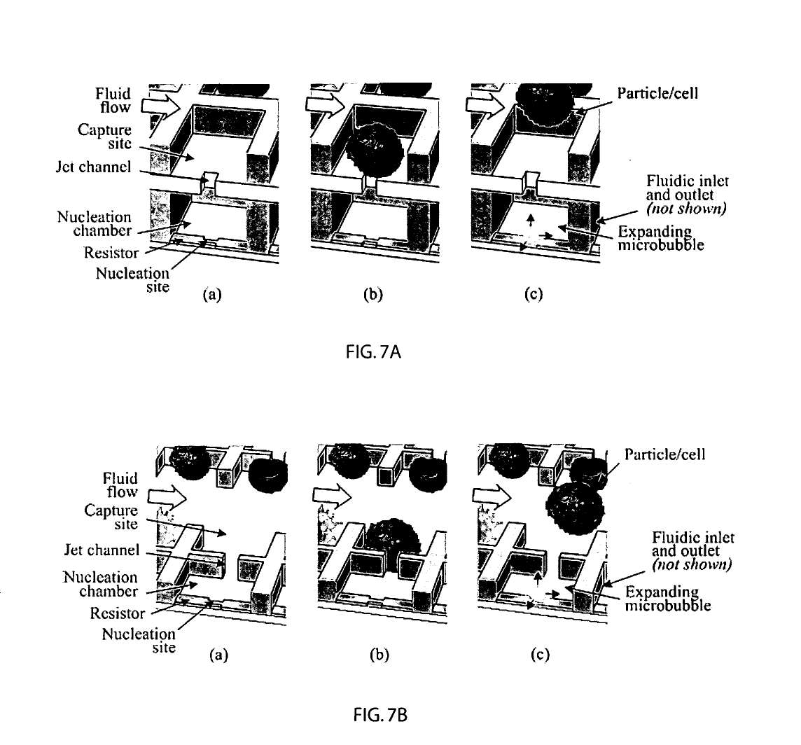

One objective of the present invention is to provide a cell analysis and sorting apparatus, which uses hydraulic forces to capture individual cells into addressable locations, and can utilize microbubble actuation to release these individual cells from their locations. In one preferred embodiment, a pressure gradient may be used to capture and maintain individual cells in the array sites, shown in FIGS. 7A and 7B. Captured cells then can be selectively released via a pulse of displaced fluid formed by a microbubble, as discussed above and as also shown in FIGS. 7A and 7B.

There are two modes of bubble nucleation: homogeneous and heterogeneous. Homogeneous nucleation occurs in a pure liquid, whereas heterogeneous nucleation, pool boiling, occurs on a heated surface at the liquid-solid interface. Under the theory of bubble nucleation, pool boiling takes place when a heater surface is submerged in a pool of liquid. As the heater surface temperature increases and exceeds the saturation temperature of the liquid by an adequate amount, vapor bubbles nucleate on the heater at suitable nucleation sites, natural or machined defects. The layer of fluid directly next to the heater is superheated, and a bubble is formed. Liquid adjacent to the newly formed bubble provides thermal energy to vaporize additional liquid at the interface between the liquid and the vapor. The bubble grows rapidly in this region, displacing equivalent volumes of liquid. The growth rate decreases dramatically when the top of the bubble extends beyond the layer of superheated liquid, where the thermal energy per unit volume is less. At the point that the bubble extends far into the cooler liquid, more hear to lost by evaporation and convection than is provided by conduction. With the inertial forces depleted, the bubble collapses, and cooler liquid flows into the newly vacated volumes. The microconvection currents flow over the defect effectively resetting the site for another nucleation.

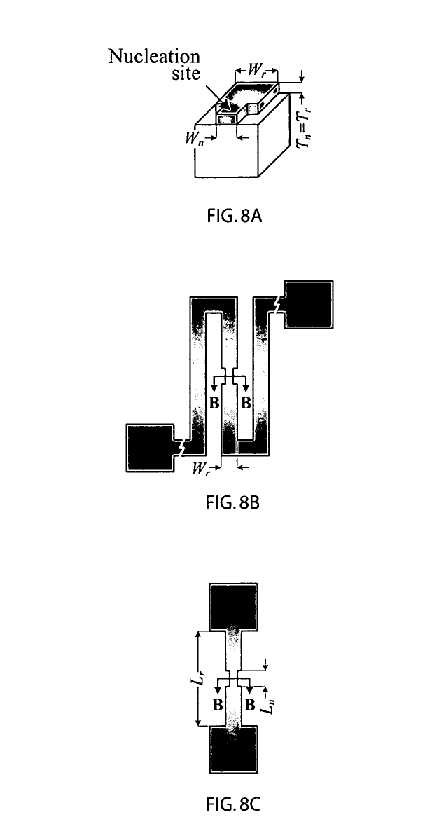

In order to heat the water to a sufficiently high temperature for microbubble formation, resistive heating elements are used. The resistive heating element can comprise a resistor typically from about 0.2 micrometers to about 0.5 millimeters wide and about 0.2 micrometers to about 5 millimeters long, and preferably at most 10 micrometers wide and at most 1500 micrometers long. In one preferred embodiment, the heating elements are planar resistive heating elements, as shown in FIGS. 8A-8C (FIG. 8A is the A-A cross-section referred to in FIGS. 8B and 8C). The planar resistive heating element can comprise a resistor with a narrowed portion preferably positioned in the center of the resistor. This narrowed portion forms the bubble nucleation site when the microbubble is formed. Typically the width of the narrowed region will range from about 1 to 99 percent of the resistor's full width, and the length of the narrowed region will range from about 1 to 99 percent of the resistor's full length. The planar resistive heating element can be formed on a surface of chamber 16 (as shown in FIGS. 4A-4D) or narrow channel 14 (as shown in FIG. 4E). The resistor can consist of a variety of geometries, including a linear or serpentine resistor.

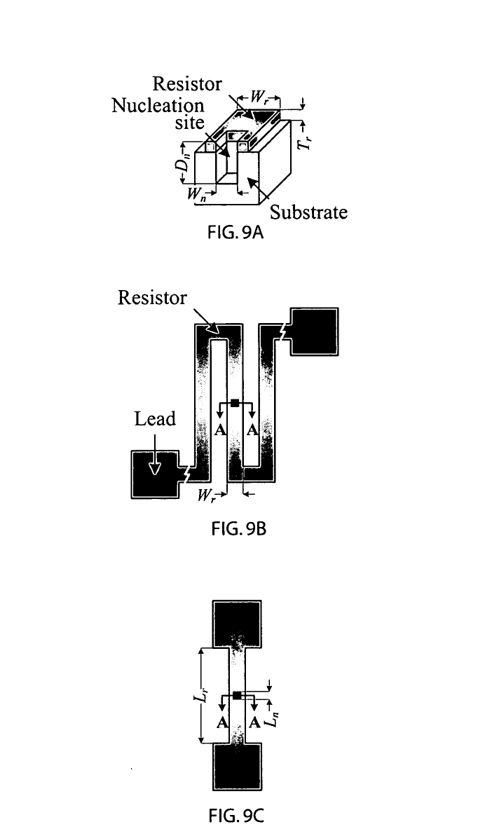

In another embodiment, the heating elements can be non-planar resistive heating elements, as shown in FIG. 9A-9C (FIG. 9A is the A-A cross-section referred to in FIGS. 9B and 9C). The bubble nucleation site in a non-planar resistive heating element is formed by a machined cavity preferably positioned through the line of horizontal symmetry, in the case of a linear resistor, or preferably positioned in the central region of the resistor, in the case of a serpentine resistor. As can be seen in FIG. 8A-8C, the non-planar resistive heating element can be formed on a surface of chamber 16 (as shown in FIGS. 4A-4D) or narrow channel (as shown in FIG. 4E), with at least one nucleation site etched into a surface of the chamber. The width of a cavity typically ranges from about 1 to 99 percent of the resistor's full width and the depth of a cavity can vary from about 0.2 micrometers to about 0.5 millimeters. The term “width” as used herein is intended to mean the diameter of a circular well or cavity or the average width in the case of other polygonal, i.e. non-circular, shapes.

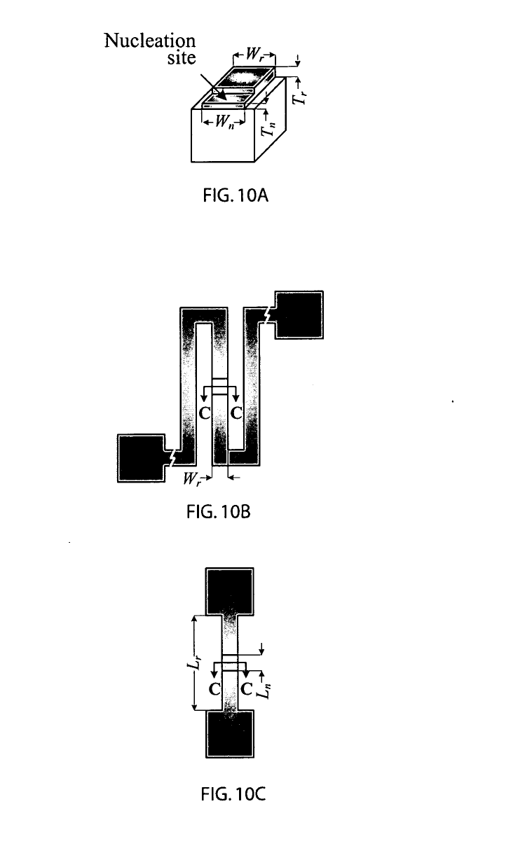

In another embodiment, the heating elements are thin-plane resistive heating elements. The bubble nucleation site is created by decreasing the height of the resistor at the horizontal line of symmetry, as shown in FIG. 10A-10C (FIG. 10A is the A-A cross-section referred to in FIGS. 10B and 10C). The step height (or height differential) will typically range from about 50 angstroms to about 10 μm and typically encompass 1 to 99 percent of the resistor's full height.

Exemplary heaters of each of these types are described in more detail below. In each instance, one design constraint is the need to keep the current density below the electromigration limit of the resistor material, while retaining an adequate degree of ohmic heating. The electromigration limit is the maximum current density which a material can endure before the atoms begin to migrate leaving the resistor inoperable.

Wells

In one embodiment of the device, square wells were micromachined into silicon in order to hold cells. A range of dimensions was chosen for these wells to allow for tests with different particle sizes and flow rates. The objective was to have the ability to trap one particle in each of an array of wells.

Well sizes ranging from 10-50 μm were chosen. Narrow channel widths of 5 μm and 8 μm were chosen since both these sizes are smaller than the minimum test particle size of 10 μm and it is necessary that particles not be able to settle down into the narrow channel. In practice, circular wells or well of other geometries can be used as well as square or rectangular wells. The actual geometry chosen will depend on the desirability of a close “fit” versus ease of manufacture. The term “width” as used herein is intended to mean the diameter of a circular well or cavity or the average width in the case of other polygonal, i.e. non-circular, shapes.

In another embodiment of the device, wells and nucleation chambers are formed by methods such as casting, hot embossing, or micromachining. Mold, cast, and/or final well and nucleation chamber materials such as SU-8 or SU-8 2000 photoresists (MicroChem Corporation, Newton, Mass.), polydimethylsiloxane (PDMS) (Sylgard 184® Silicone Elastomer, Dow Coming Corporation, Midland, Mich.), etched silicon, glass, plastic, UV curable polymers, and biomaterials may be used in the process. Other techniques and materials obvious to those skilled in the art may be implemented to form the structures. Additionally, the surface(s) of the structure(s) may be engineered to have different surface chemistries.

A range of dimensions were chosen for the wells to enable each capture site to hold one or multiple cells. Well dimensions may vary depending on the object of capture, with widths and depths ranging from about 0.2 micrometers to about 1 millimeter. In an embodiment of the design geometrically similar to FIG. 7A, each well had a diameter of 30 μm and height of 15 μm. Each nucleation chamber has dimensions of 400 μm in length and 300 μm in width and height. In an embodiment of the design geometrically similar to FIG. 7B, each well, nucleation chamber, and narrow channel had a height of 20 μm. Wells were configured in circular and rectangular geometries, though additional geometries can be used in practice. In kind to the silicon well manufacture specifications, the practical geometries will depend on the desirability of a close “fit” versus ease of manufacture.

In one molding and casting embodiment, PDMS molds are fabricated on 150 mm diameter silicon wafers (Wafernet, Inc., San Jose, Calif.). In an embodiment of the device geometrically similar to FIG. 7A, one mold defines the nucleation chambers and the second fluid flow header. A second mold defines the wells, narrow channels, and the header in which particles flow.

After a piranha clean, custom alignment marks optimized for viewing through thick layers of photoresist are patterned using standard positive photolithography techniques. Alignment marks are etched in a deep trench etcher system. After a second piranha clean, the wafers are dehydrated serially on a hot plate or in parallel in a convection oven.

To form the nucleation chamber mold, a polyimide coater is used to spin on 6 μm of negative resist (SU-8 2005, MicroChem Corporation, Newton, Mass.) on each etched wafer. The resist is soft baked, exposed on a mask aligner, and postbaked. Next, a three-layer process is used to deposit a total of 300 μm of negative resist (SU-8 50, MicroChem Corporation, Newton, Mass.; SU-8 2075, MicroChem Corporation, Newton, Mass.). The coater is used to spin on 100 μm of resist, which is then soft baked. This two step process is repeated thrice at which point the 300 μm of photoresist is air dried and then baked in a convection oven on a metal plate until hard. The photoresist is then exposed on a mask aligner, postbaked, and developed (SU-8 Developer, MicroChem Corporation, Newton, Mass.). An isopropanol rinse and nitrogen dry complete the DI mold fabrication process.

It should be understood that the term “depositing” is meant to include spinning, laminating, spraying, or any other method of depositing a substance onto a surface.

To form the well mold, a three-layer process, identical to that of the nucleation chamber mold, is used to deposit 300 μm of photoresist on each etched wafer. Then, 15 μm of negative resist (SU-8 2010, MicroChem Corporation, Newton, Mass.) is spun, soft baked, exposed, and postbaked.

After a convection oven bake on a metal plate until hard, the coater is used to spin on 50 μm of negative resist (SU-8 50, MicroChem Corporation, Newton, Mass.). The resist is soft baked, exposed, and postbaked. The photoresist is then developed. An isopropanol rinse and nitrogen dry complete the capture site mold fabrication process.

In an embodiment of the device geometrically similar to FIG. 7B, one mold defines the nucleation chambers, fluid flow header, wells, and narrow channels. A coater spins on 20 μm of negative resist (SU-8 2015, MicroChem Corporation, Newton, Mass.). The resist is soft baked, exposed on a mask aligner, postbaked, and developed (SU-8 Developer, MicroChem Corporation, Newton, Mass.). An isopropanol rinse and nitrogen dry complete the DI mold fabrication process.

Casts are formed by pouring the PDMS over the fabricated molds and curing. The PDMS casts are then cut into chips and aligned to the heaters. For the embodiment geometrically similar to FIG. 7A, a glass slide or blank PDMS cast forms the upper surface of the header in which the particles flow. Surface activation in an RF plasma cleaner/sterilizer unit is used for bonding where applicable.

Design and Fabrication of Resistive Heaters

Out-of-plane, in-plane, and thin-plane microbubble nucleation sites can all serve as engineered defects to enable mono-nucleation of microbubbles. The term “defect” as used herein is intended to mean an engineered nucleation site that has been designed with the purpose of serving to enable mono-nucleation of microbubbles.

For an out-of-plane microbubble generator, a machined cavity through the central region of a serpentine, folded, resistor can serve as a nucleation site, effectively providing a defect while creating a region of higher resistance. Alternatively, an out-of-plane microbubble generator can be formed by a machined cavity through the line of horizontal symmetry in a linear resistor. The out-of-plane geometry is shown in FIGS. 9A-9C with resistor dimensions of length Lᵣ by width Wᵣ by thickness Tᵣ and cavity dimensions of length Lₙ by width Wₙ by depth Dₙ.

Reducing the cross sectional area of the resistor at the line of horizontal symmetry, effectively increasing the resistor resistance in that region, forms in-plane and thin-plane nucleation sites. Narrowing the resistor at the midpoint forms a nucleation site in the plane of the resistor for an in-plane microbubble generator shown in FIGS. 8A-8C with resistor dimensions Lᵣ by Wᵣ by Tᵣ and nucleation site dimensions Lₙ by Wₙ by thickness Tₙ where Tᵣ=Tₙ. Decreasing the height of the resistor at the horizontal line of symmetry creates the nucleation site of a thin-plane microbubble generator shown in FIGS. 10A-10C.

FIGS. 10A-10C illustrate the thin-plane resistor with resistor dimensions Lᵣ by Wᵣ by Tᵣ and nucleation site dimensions Lₙ by Wₙ by Tₙ where Tₙ≠Tᵣ, the thickness of the resistor.

The range or resistances of the nucleation sites and the total resistor resistances can be calculated using

$$R = \frac{L}{TW} {ρ}_{e} \qquad ( 8 )$$

where L is the resistor length and direction in which current flows; W is the resistor width; T is the resistor thickness, and ρₑ is the electrical resistivity of the material. The equations to calculate the resistances for each nucleation site design are

$$R = ( \frac{( {L}_{r} - {L}_{n} )}{{T}_{r} {W}_{r}} + \frac{2 {L}_{n}}{{T}_{r} ( {W}_{r} - {W}_{n} )} ) {ρ}_{e} \qquad ( 9 )$$

for the out-of-plane design,

$$R = ( \frac{( {L}_{r} - {L}_{n} )}{{T}_{r} {W}_{r}} + \frac{{L}_{n}}{{T}_{r} {W}_{n}} ) {ρ}_{e} \qquad ( 10 )$$

for the in-plane design, and

$$R = ( \frac{( {L}_{r} - {L}_{n} )}{{T}_{r} {W}_{r}} + \frac{{L}_{n}}{{T}_{n} {W}_{r}} ) {ρ}_{e} \qquad ( 11 )$$

for the thin-plane design.

The resistance of the power lead for each resistor is preferably designed to be at least a factor of ten less resistive than the resistor. The effect of the length of the lead on the resistance of the lead can be examined by comparing the ratio of the length and width for each resistor length. In one embodiment of the invention, there are two lead lengths used. The first L/W ratio was 4.67, and the second L/W ratio was 5.22. Using Equation (8) and the electrical resistivity of platinum, the lead resistance equaled approximately 5 Ω, and the variation in the resistance between the leads was less than 1 Ω. Thus, the resistance of each lead is less than 10 percent of the resistor resistance for resistors with at least a 50 Ω resistance.

Out-of-Plane Resistor Fabrication

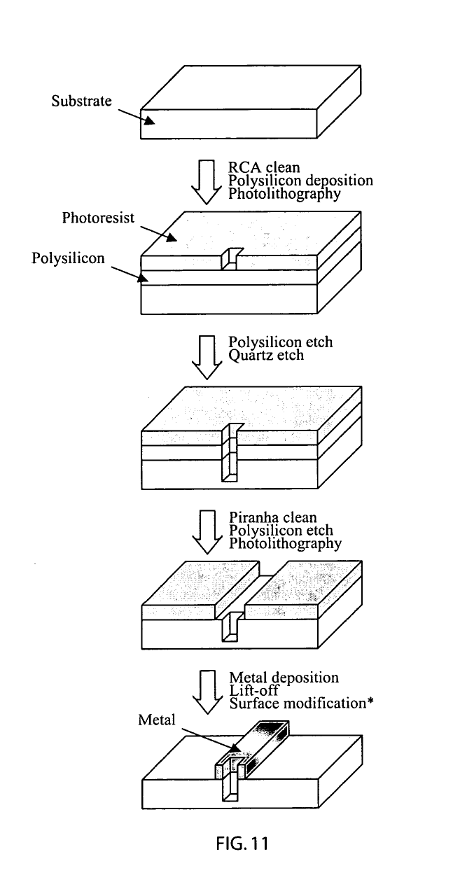

In one embodiment of the device, out-of-plane resistors can be fabricated on 150 mm diameter quartz wafers (Mark Optics, Inc., Santa Ana, Calif.). Other optically transparent substrates such as glass wafers (Pyrex 7740, Mark Optics; Borofloat, Mark Optics, Inc.) also may be used. However, substitute substrate viability is limited by available etching technologies, as fabrication requires etching a nucleation cavity. A schematic of the out-of-plane resistors is shown in FIG. 9, and the process flow is shown in FIG. 11.

After an RCA clean of the quartz substrates, 2 μm of polysilicon is deposited by a pyrolysis of silane (SiH₄) in a low pressure chemical vapor deposition (LPCVD) reactor. The polysilicon layer serves as an etch mask later in the process. Nucleation sites are patterned on the polysilicon using standard positive photolithography techniques, as shown in FIG. 8B. The polysilicon mask is formed by etching through the 2 μm of polysilicon in a deep trench etcher system. For this wafer lot, the mask then is used to etch the 6 μm diameter by 16 μm deep cylindrical cavities in the quartz. Surface Technology Systems (STS) performed a proprietary quartz wafer etch for this process step. See FIG. 9A for cavity detail.

After piranha cleaning, the polysilicon mask is removed in a polysilicon etcher. Metal is patterned using standard image reverse photolithography techniques, illustrated in FIG. 11. An evaporative deposition system successively deposits a 100 Å titanium adhesion layer and 1,000 Å platinum. After metallization, excess metal is lifted off in an acetone bath. To enable device reliability comparison, a portion of the wafer lot is annealed in an atmospheric diffusion tube with nitrogen. Some chip surfaces are modified using silane (tridecafluoro-1,1,2,2-tetrahydrooctyl-1-triethoxysilane, United Chemical Technologies, Bristol, Pa.), which makes the surfaces more hydrophobic. A chip is silanized by pumping a 2% solution of silane in ethanol through the packaged μBA device. The solution is allowed to remain stagnant in the channels for 60 s before the system is flushed with ethanol.

In-Plane Resistor Fabrication

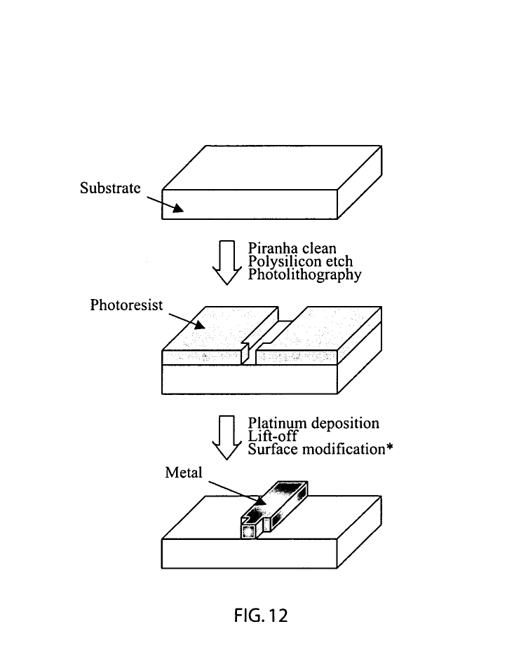

In one embodiment of the device, in-plane resistors are fabricated on 150 mm diameter fused silica, quartz wafers. The process flow is illustrated in FIG. 12. After a piranha clean, the metal mask is patterned using standard image reverse photolithography techniques, as shown in FIG. 12. An evaporative deposition system successively deposits a 100 Å titanium adhesion layer and a 1,000 Å platinum layer. After metallization, excess metal is lifted off in an acetone bath. The wafers are cut into chips with a diesaw. To enable device reliability comparisons, a portion of the wafer lot is annealed in an atmospheric diffusion tube with nitrogen and/or surface modified in the same manner as the out-of-plane resistors.

Thin-Plane Resistor Fabrication

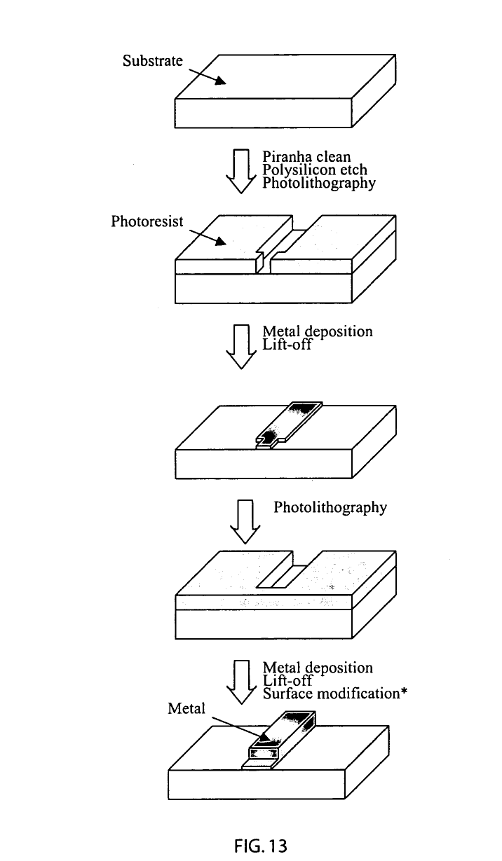

In one embodiment of the device, thin-plane resistors are fabricated on 150 mm diameter fused silica, quartz wafers. The process flow is illustrated in FIG. 13. After a piranha clean, the metal mask is patterned using standard image reverse photolithography techniques, as shown. An evaporative deposition system successively deposits a 100 Å titanium adhesion layer and a 50-950 Å platinum layer. Excess metal is lifted off in an acetone bath, as depicted in FIG. 13. The second metal mask is patterned using image reverse photolithography, as shown. The evaporative deposition system deposits a 50-900 Å platinum layer after which excess metal is lifted off in an acetone bath. An alternative to the two-step formation of an evaporative film would be electrodeposition. After metallization, the wafers are cut into chips with a diesaw. To enable device reliability comparisons, a portion of the wafer lot is annealed in an atmospheric diffusion tube with nitrogen and/or surface modified in the same manner as the out-of-plane resistors.

### EXAMPLES AND RESULTS

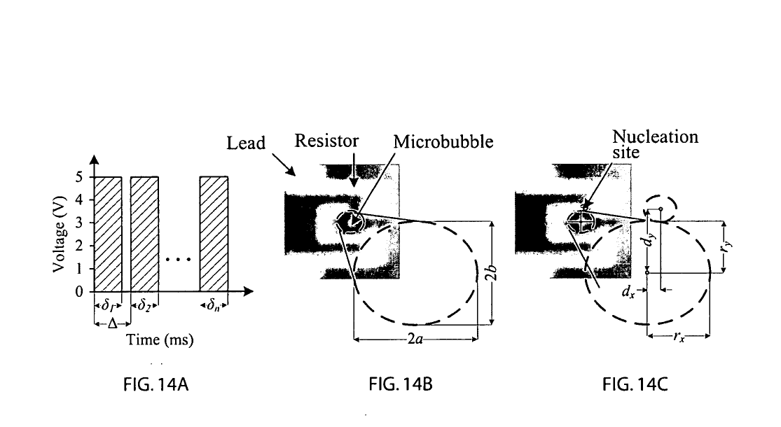

Two system input patterns are used in performance testing—standard input and chirped input. Both input patterns have pulse height 5 V, pulse width δ, and are repeated with frequency 1/Δ, as shown in FIG. 14A. For standard input, δ is a fixed value, meaning δ₁=δ₂= . . . =δₙ. For chirped input, δ increments in value by a constant Δδ, meaning δₙ₊₁=δₙ+Δδ. For example, for δ₁=1 ms and Δδ=0.5 ms, δ₂=1.5 ms, δ₃=2 ms, . . . For both input types, 0.125 ms≦δ≧50 ms, and 1 s≦Δ≧15 min or Δ=∞, meaning no repeated pulse.

For each microbubble, the average diameter D_{avg} is measured along the major and minor axes of the microbubble over the duration of the dissipation process. D_{avg} is defined as

$${D}_{avg} = \frac{2 a + 2 b}{2} \qquad ( 12 )$$

where a is the length of the semi-major axis, and b is the length of the semi-minor axis, as shown in FIG. 14B. The maximum D_{avg} is defined as the largest measured D_{avg} for a given response.

Eccentricity e is a parameter used in mathematics and astronomy to measure deviation of a conic section from circularity or the ellipticity of an object. This parameter quantifies the shape of an object and is defined as

$$e = \sqrt{1 - \frac{{b}^{2}}{{a}^{2}}} \qquad ( 13 )$$

where a is the length of the semi-major axis, and b is the length of the semi-minor axis. As a point of reference, a perfect circle would have e=0. An ellipse would have 0<e<1. An eccentricity measurement is taken as shown in FIG. 14B.

Centricity c is a constant used to quantify the deviation of the center of a circle or ellipse from a designated point. The centricity is defined as

$${c}_{x} = \frac{{d}_{x}}{{r}_{x}} \qquad ( 14 )$$

and

$${c}_{y} = \frac{{d}_{y}}{{r}_{y}} \qquad ( 15 )$$

where d is the distance from the center of the nucleation site to the center of the microbubble in the x- or y-direction, and r is the radius of the microbubble. As a point of reference, a perfectly centered microbubble would have cₓ=c_{y}=0. A microbubble with a left edge at the nucleation site and centered in the y-direction has cₓ=1 and c_{y}=0. A centricity measurement is taken as shown in FIG. 14C.

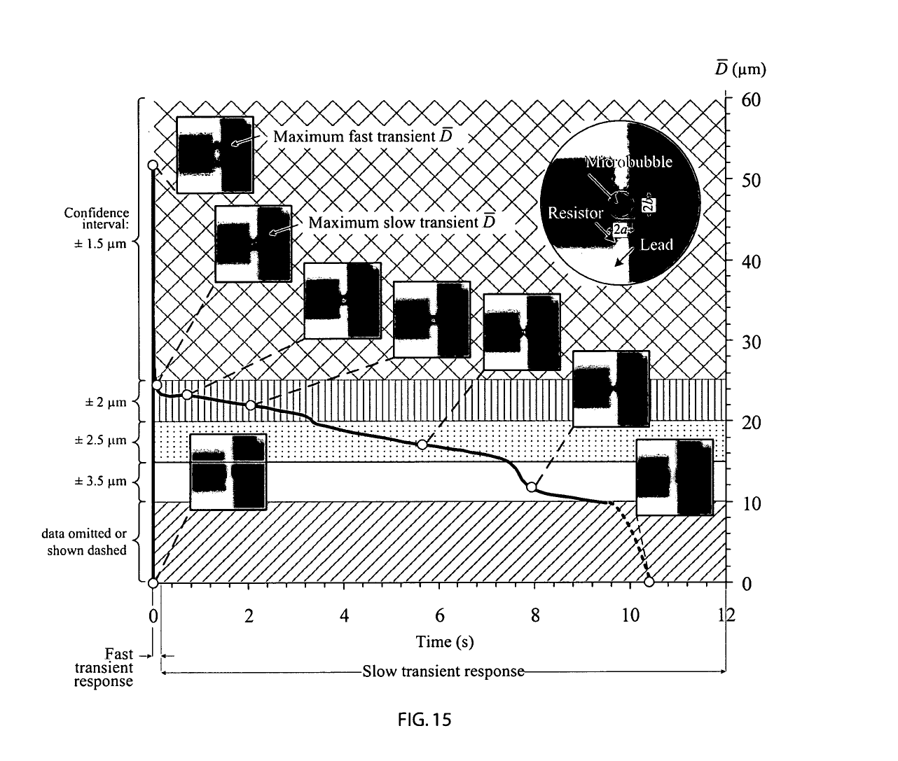

A typical complete system response to standard pulse input of width δ=30 ms and Δ=∞ at critical points along the dissipation curve was determined, shown in FIG. 15. The complete system response consists of a fast transient response and a slow transient response. The fast transient response demonstrates nucleation. The slow transient response includes the remainder of the data as the microbubble dissipates.

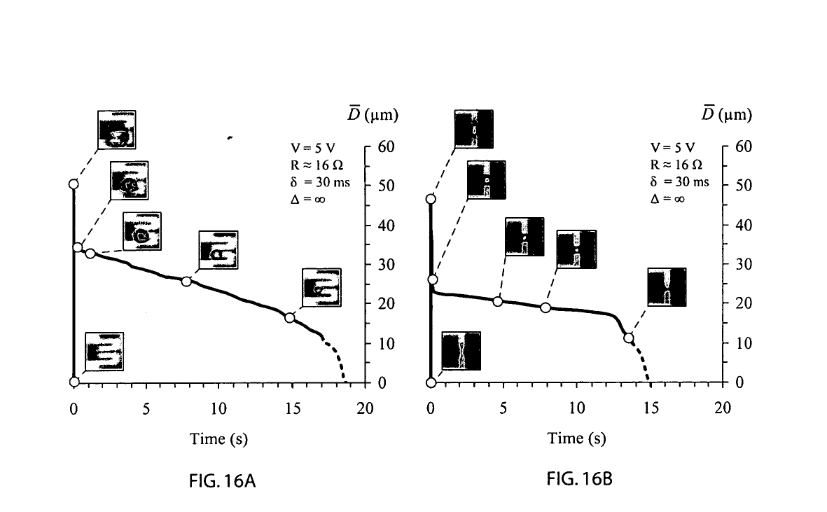

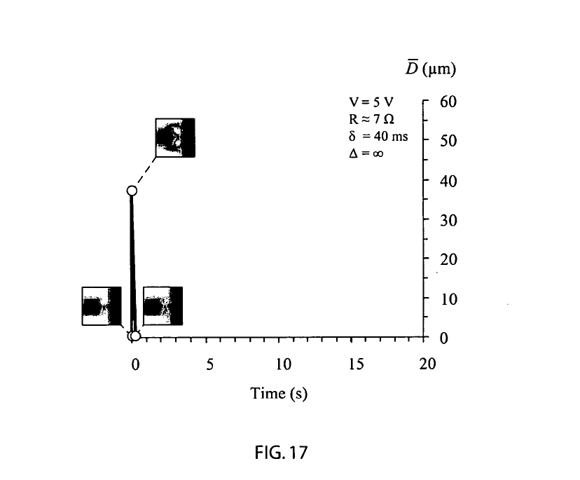

The out-of-plane nucleation site resistors nucleated single microbubbles per pulse for all tested lengths Lᵣ<1270 μm. The in-plane nucleation site resistors were successful mono-bubble nucleators for geometries with Lᵣ≦108 μm. Typical complete system responses to a single pulse of voltage applied to an out-of-plane and in-plane actuator at time t=0 s are shown in FIGS. 16A and 16B. As Lᵣ decreases to lengths such as 10 μm with sufficiently small pulses applied, only a fast transient is evident as shown in FIG. 17.

Performance testing over a representative range of the microbubble actuation (μBA) geometries was used to form a comparison of nucleation techniques. For example, one comparison included one out-of-plane resistor and three in-plane resistors: an out-of-plane nucleation site resistor with 6 Am diameter nucleation cavity with a hydrophobic surface modification of CYTOP™ and silane to enable repeatable nucleation at the nucleation site and three representative in-plane resistors with no surface modifications, nucleation site widths of 3 μm, and lengths of 10, 20, and 30 μm, respectively. A chirped input was used with 10 ms≦δ≧50 ms, Δδ=10 ms, and fΔ≈4 mHz.

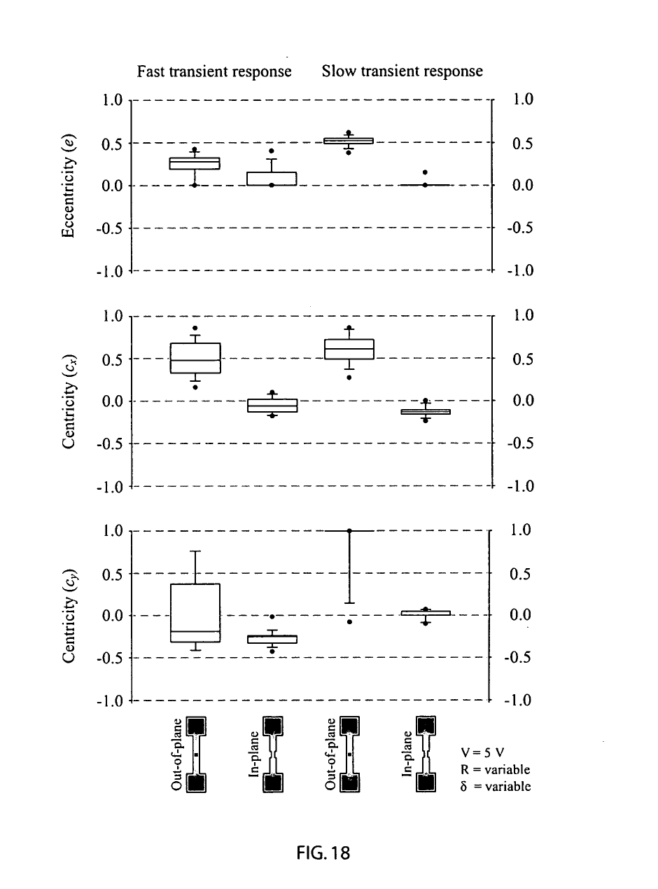

FIG. 18 shows the fast and slow transient response for out-of-plane and in-plane resistors. The fast transient response of the out-of-plane geometry was more elliptical than spherical, as e≠0. In contrast, the fast transient responses of the in-plane geometries were more spherical. The slow transient response of the out-of-plane geometry has an eccentricity represented in a tighter box plot and is more elliptical than spherical with a mean e≈0.5. The slow transient in-plane resistors generate tight data, with spherical bubbles of mean e≈0.

Regarding centricity, the fast transient response of the out-of-plane geometry has means of cₓ≈0.5 and c_{y}≈−0.2, where a centered microbubble would have a mean value of cₓ≈c_{y}≈0. The in-plane geometries demonstrate a fast transient response with mean values closer to centered in both x- and y-directions. Similar results were seen for the slow transient responses of the out-of-plane and in-plane geometries with tighter data in both instances.

Referring again to FIG. 18, the out-of-plane geometry exhibited an off-center slow transient response. The cₓ and c_{y} box plot heights demonstrate that the location of the microbubble center varied. In contrast, the in-plane geometry had a cₓ and c_{y} repeatable, relatively centered, slow transient response.

For symmetrical in-plane resistors, statistical results showed that slow transient responses were spherical in shape. Additionally, a non-symmetric out-of-plane resistor exhibited an elliptically-shaped transient response. However, a linear resistor with an out-of-plane nucleation site generated spherical microbubbles. Thus, the symmetry of the resistor affects the resultant shape of the microbubble, a conclusion that also is supported by out-of-plane and in-plane modeling.

The potential relationship between geometry and available hot-adjacent liquid during the bubble growth phase further suggests that the symmetry of the microfabricated geometry does have an effect on the resultant shapes of the slow and fast transient responses. By carefully designing the geometry of the resistor, the results show that a dependably spherical microbubble can be nucleated. A potential for engineering the shape of the early slow transient microbubble may also exist.

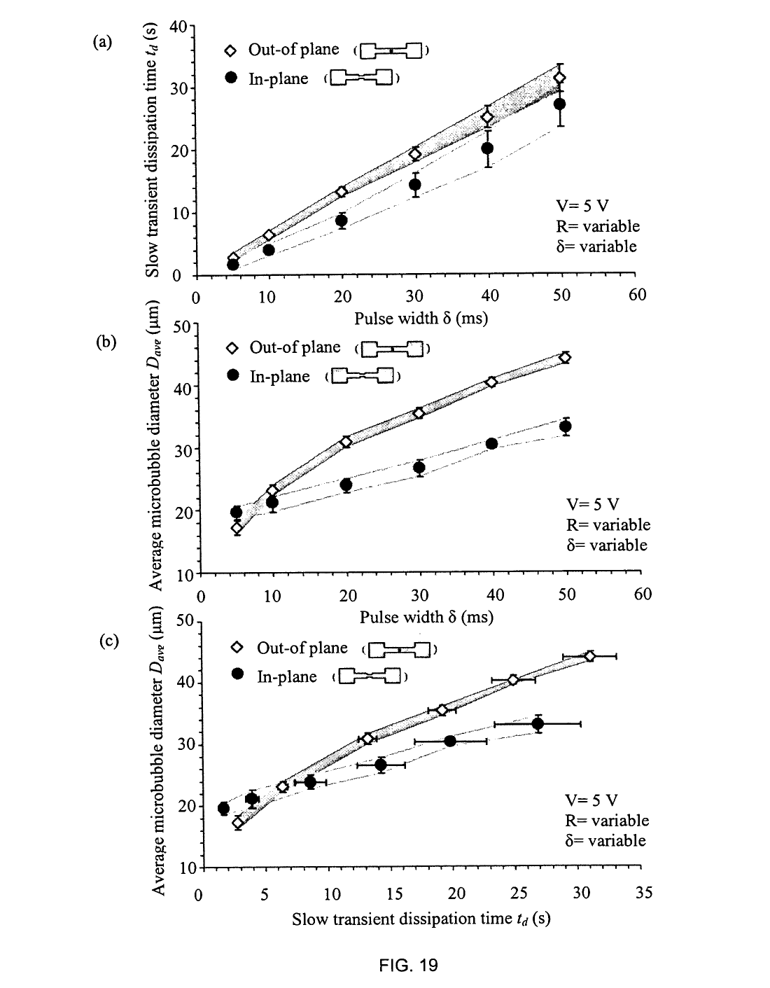

For in-plane and out-of-plane resistors, the slow transient maximum D_{avg} increases as input energy increases. As illustrated in FIG. 19, increase in input energy can be attributed to the geometry of the resistor or the use of a lower resistance resistor or a larger δ. The correlation between increased energy input and increased slow transient maximum D_{avg} output may be due to the available hot-adjacent liquid at the liquid-vapor interface.

From microbubble theory, liquid adjacent to the nucleated bubble serves as a growth factor. The hot adjacent liquid provides thermal energy to vaporize more liquid at the liquid-vapor interface. Thus, the size of the slow transient maximum D_{avg} is a function of the available energy. Increasing the regional amount of thermal energy available then would make more thermal energy available for the vaporization process. The outcome would be a larger slow transient maximum D_{avg}. Thus, the slow transient maximum D_{avg} is a function of the input energy to the system. By engineering the amount of energy available to the microbubble in the growth phase, the slow transient maximum D_{avg} can be regulated to within the confidence interval and to system specifications as long as drift is controlled.

A larger microbubble contains more vaporized liquid within its volume. Since the evaporation and convection losses occur over the surface area of the microbubble, a microbubble of larger volume would require longer to dissipate. The results demonstrate that dissipation time is related to the energy input to the system and is a function of the slow transient maximum D_{avg}. Regulating the slow transient maximum D_{avg} to within the confidence interval and to system specifications by controlling the input energy enables simultaneous regulation of the dissipation time as long as drift is controlled.

Differences in out-of-plane and in-plane resistor geometries range from fabrication steps, substrates, and post-fabrication surface modifications to required chip size and microbubble performance. The out-of-plane resistor geometry requires two masks to etch the nucleation sites and define the resistors. The in-plane resistor geometry requires one mask to define both the resistors and nucleation sites.

Without an etch step in the in-plane fabrication process, resistor geometries can be fabricated on a variety of optically transparent substrate that allow data to be acquired from both vertical axes. The μBA-powered ILDAC standard has been fused silica (quartz), as quartz is an etchable substrate. For in-plane geometries, several less expensive glass substitutes such as autoclavable Pyrex and Borofloat may be used.

Previous research on out-of-plane, cavity-sponsored nucleation demonstrated that a surface modification such as CYTOP™ or silane is required to nucleate bubbles repeatedly at a nucleation site. In contrast, in-plane geometries require no surface modifications for successful and repeatable microbubble nucleation. For some applications, chip size can be an issue. Typical out-of-plane resistors occupy areas on the order of 1,000 to 10,000 μm². Depending on the output transient desired, in-plane resistor designs can occupy areas on the order of 100 μm².

The shape and location of out-of-plane microbubbles vary over the course of multiple trials. Seeming to nucleate almost randomly around the nucleation site, out-of-plane generated microbubbles range from the most common shape, elliptical, to occasionally spherical. The elliptical microbubbles often become spherical several seconds into the slow transient dissipation process. In comparison, the in-plane generated microbubble is spherical and centered on the nucleation site.

The out-of-plane and in-plane geometries also share some attributes. Both geometries exhibit the same functional maximum slow transient D_{avg} dependence on input energy. The out-of-plane and in-plane geometries also evince the same functional t_{d} dependence on the maximum slow transient D_{avg} and exhibit a similar functional relationship between t_{d} and the input energy.

All publications cited herein are incorporated in their entirety by reference.

While the invention has been particularly shown and described above with reference to several preferred embodiments and variations thereon, it is to be understood that additional variations could be made in the invention by those skilled in the art while still remaining within the spirit and scope of the invention, and that the invention is intended to include any such variations, being limited only by the scope of the appended claims.

What is claimed is:

1. A cell manipulation apparatus comprising:
an array of sites arranged across a substrate in a pattern, each site configured to hold one cell, wherein each site further comprises:
a cell capture mechanism associated with each site that is capable of capturing the cell, and
a cell release mechanism comprising at least one microbubble actuator for selectively releasing the cell from the site.

2. The apparatus of claim 1, wherein the capture mechanism comprises a geometric well associated with each site having a width of about 0.2 micrometers to about 50 micrometers.

3. The apparatus of claim 1, wherein the capture mechanism comprises a geometric well associated with each site having a width of about 0.2 micrometers to about 1 millimeter.

4. The apparatus of claim 1, wherein the capture mechanism comprises a geometric well associated with each site having a depth of about 0.2 micrometers to about 50 micrometers.

5. The apparatus of claim 1, wherein the capture mechanism comprises a geometric well associated with each site having a depth of about 0.2 micrometers to about 1 millimeter.

6. The apparatus of claim 1, wherein the capture mechanism comprises a geometric well associated with each site having sufficient vertical depth to hold the cell by gravitational force.

7. The apparatus of claim 1, wherein the capture mechanism comprises at least one fluid flow path coupled to each site to hold the cell by a fluid pressure gradient.

8. The apparatus of claim 1, wherein the capture mechanism comprises a non-uniform electric field trap to hold the cell by electrostatic force.

9. The apparatus of claim 1, wherein the release mechanism further comprises a chamber in fluid communication with the well, in which at least one microbubble can be formed to apply an ejective force to the well.

10. The apparatus of claim 1, wherein the release mechanism further comprises at least one resistive heating element capable of initiating microbubble formation.

11. The apparatus of claim 10, wherein the at least one resistive heating element is aligned with at least one surface of a flow path coupled to the well.

12. The apparatus of claim 10, wherein the at least one resistive heating element is a linear resistor.

13. The apparatus of claim 10, wherein the at least one resistive heating element is a serpentine resistor.

14. The apparatus of claim 10, wherein the width of the at least one resistive heating element ranges from about 0.2 micrometers to about 0.5 millimeters.

15. The apparatus of claim 10, wherein the width of the at least one resistive heating element is about 0.2 micrometers to about 50 micrometers.

16. The apparatus of claim 10, wherein the length of the at least one resistive heating element ranges from about 0.2 micrometers to about 5 millimeters.

17. The apparatus of claim 10, wherein the length of the at least one resistive heating element ranges from about 0.2 micrometers to about 1500 micrometers.

18. The apparatus of claim 1, wherein the release mechanism is a microfluidic actuator comprising at least one resistor with at least one bubble nucleation site formed along its length by at least one narrowing of an electrical conductive path.

19. The apparatus of claim 1, wherein the release mechanism is a microfluidic actuator comprising at least one resistor with at least one bubble nucleation site formed along its length by at least one narrowing of the resistor's width.

20. The apparatus of claim 19, wherein the width of the at least one narrowed region ranges from about 0.2 micrometers to about 0.5 millimeters.

21. The apparatus of claim 19, wherein the width of the at least one narrowed region ranges from about 0.2 micrometers to about 50 micrometers.

22. The apparatus of claim 19, wherein the width reduction ranges from about 1 to about 99 percent of the resistor's width.

23. The apparatus of claim 19, wherein the length of the at least one narrowed region ranges from about 0.2 micrometers to about 5 millimeters.

24. The apparatus of claim 19, wherein the length of the at least one narrowed region ranges from about 0.2 micrometers to about 1,500 micrometers.

25. The apparatus of claim 19, wherein the length of the at least one narrowed region ranges from about 1 to about 99 percent of the resistor's length.

26. The apparatus of claim 1, wherein the release mechanism is a microfluidic actuator comprising at least one resistor with at least one bubble nucleation site formed along it length by at least one reduction of the resistor's height.

27. The apparatus of claim 26, wherein the height reduction ranges from about 50 angstroms to about 10 micrometers.

28. The apparatus of claim 26, wherein the height reduction ranges from about 1 to about 99 percent of the resistor's height.

29. The apparatus of claim 1, wherein the release mechanism is a microfluidic actuator comprising at least one resistor with at least one bubble nucleation site formed along it length by at least one physical defect.

30. The apparatus of claim 29, wherein the at least one defect is a cavity formed in the resistor.

31. The apparatus of claim 30, wherein the at least one cavity's depth ranges from about 0.2 micrometers to about 0.5 millimeters.

32. The apparatus of claim 30, wherein the at least one cavity's depth ranges from about 0.2 micrometers to about 50 micrometers.

33. The apparatus of claim 30, wherein the at least one cavity's width ranges from about 0.2 micrometers to about 0.5 millimeters.

34. The apparatus of claim 30, wherein the at least one cavity's width ranges from about 0.2 micrometers to about 50 micrometers.

35. The apparatus of claim 30, wherein the at least one cavity's width ranges from about 1 to about 99 percent of the resistor's width.

36. The apparatus of claim 1, wherein the release mechanism is a microfluidic actuator selected from the group comprising of an at least one resistor with at least one bubble nucleation site formed along its length by at least one narrowing of the resistor's width, at least one resistor with at least one bubble nucleation site formed along it length by at least one reduction of the resistor's height, and at least one resistor with at least one bubble nucleation site formed along it length by at least one physical defect.

37. The apparatus of claim 1, wherein each site has a unique address and is independently controllable.

38.-39. (canceled)

40. The apparatus of claim 1, the apparatus further comprising a fluid introducing element for introducing a gradient of fluid across at least a portion of a population of captured cells.

41. The apparatus of claim 1, the apparatus further comprising a fluid introducing elements for introducing a plurality of distinct fluids across at least a portion of a population of captured cells.

42. A method of making a cell manipulating apparatus, comprising the steps of:
forming a well on one surface of a substrate, the well being configured and dimensioned to hold one cell;
forming a bubble nucleation chamber on the same substrate or a second substrate;
forming a channel on the same substrate or the second substrate to connect the well and chamber together and permit fluid communication therebetween; and
coupling a heating element to the bubble nucleation chamber.

43. The method of claim 42, wherein the method further comprises etching at least one substrate to form the well, channel and chamber.

44. The method of claim 42 wherein the first substrate is a silicon wafer and the steps of etching further comprise:
growing thermal oxide onto a first surface of a the silicon wafer substrate;
patterning the oxide using a first mask that defines the shape of the well;
spinning photoresist on top of the oxide;
patterning the oxide using a second mask that defines the shape of the channel;
etching the wafer to form the channel using the second mask;
etching the wafer to form the well using the first mask;
depositing photoresist on an opposite surface of the silicon wafer substrate;
patterning the photoresist using a third mask that defines the shape of the chamber; and
etching the wafer to form the chamber, the chamber having sufficient depth to connect with the channel.

45. The method of claim 42 wherein the step of forming the at least one heating element further comprises forming a resistive heating element on the same or the second substrate and coupling the at least one heating element to the bubble nucleation chamber.

46. The method of claim 45, wherein the step of forming the at least one heating element comprises:
forming at least one conductor on the same or the second substrate.

47. The method of claim 46, wherein the step of forming the at least one conductor further comprises:
spinning photoresist onto the same or the second substrate;
patterning the photoresist with a mask that defines the shape of the at least one conductor;
evaporating at least one metal onto the same or the second substrate; and
selectively removing the metal from the substrate.

48. The method of claim 46, wherein the step of forming the at least one conductor further comprises:
spinning photoresist onto the same or the second substrate;
patterning the photoresist with a mask that defines the shape of the at least one conductor;
evaporating at least one metal onto the same or the second substrate;
selectively removing the metal from the substrate;
spinning photoresist onto the same or the second substrate;
patterning the photoresist with a mask that defines the shape of the at least one conductor;
evaporating at least one metal onto the same or the second substrate; and
selectively removing the metal from the substrate.

49. The method of claim 46, wherein the step of forming the at least one heating element further comprises patterning a conductive material to define at least one linear resistor.

50. The method of claim 46, wherein the step of forming the at least one heating element further comprises patterning a conductive material to define at least one serpentine resistor.

51. The method of claim 46, wherein the step of forming the at least one heating element further comprises patterning a conductive material to define at least one resistor with at least one narrowed region that can serve as at least one bubble nucleation site.

52. The method of claim 46, wherein the method further comprises forming at least one resistor with at least one thinned region that can serve as at least one bubble nucleation site.

53. The method of claim 46, wherein the method further comprises forming at least one resistor with at least one defect that can serve as at least one bubble nucleation site.

54. The method of claim 46, wherein the method further comprises forming at least one resistor with at least one cavity that extends through the at least one resistor that can serve as at least one bubble nucleation site.

55. The method of claim 46, wherein the method further comprises forming at least one resistor with at least one cavity that extends through the at least one resistor and into the same substrate or the second substrate as at least one out-of-plane cavity that can serve as at least one bubble nucleation site.

56. The method of claim 46, wherein the method further comprises forming at least one resistor with the method selected from the group comprising of patterning a conductive material to define at least one resistor with at least one narrowed region that can serve as at least one bubble nucleation site, at least one thinned region that can serve as at least one bubble nucleation site, at least one defect that can serve as at least one bubble nucleation site, and at least one cavity that extends through the at least one resistor that can serve as at least one bubble nucleation site.

57. The method of claim 42, wherein the method further comprises sealing the apparatus.

58. The method of claim 42, wherein the method further comprises making at least one mold to form the well, channel and chamber.

59. The method of claim 42, wherein the method further comprises machining at least one material to form the well, channel and chamber.

60. The method of claim 42, wherein the method further comprises depositing at least one material to form the well, channel and chamber.

61. The method of claim 58 wherein the first substrate is a silicon wafer and the steps of forming the mold further comprise:
depositing photoresist onto the surface of a silicon wafer substrate;
patterning the photoresist using a first mask that defines alignment marks;
etching the wafer to form the alignment marks;
depositing photoresist onto the surface of the silicon wafer substrate;
patterning the photoresist using a second mask that defines at least the header in which the particles flow;
depositing photoresist onto the surface of the silicon wafer substrate;
patterning the photoresist using a third mask that defines at least the wells; and
developing the photoresist mold structure.

62. The method of claim 58 wherein the second substrate is a silicon wafer and the steps of forming the mold further comprise:
depositing photoresist onto the surface of a silicon wafer substrate;
patterning the photoresist using a first mask that defines alignment marks;
etching the wafer to form the alignment marks;
depositing photoresist onto the surface of the silicon wafer substrate;
patterning the photoresist using a second mask that defines at least part of the chambers and fluid flow channels connected to the chamber;
depositing photoresist onto the surface of the silicon wafer;
patterning the photoresist using a third mask that defines at least part of the chambers; and
developing the photoresist mold structure.

63. The method of claim 58 wherein the second substrate is a silicon wafer and the steps of forming the mold further comprise:
depositing photoresist onto the surface of a silicon wafer substrate;
patterning the photoresist using a first mask that defines alignment marks;
etching the wafer to form the alignment marks;
depositing photoresist onto the surface of the silicon wafer substrate;
patterning the photoresist using a second mask that defines at least part of the chambers and fluid flow channels connected to the chamber; and
developing the photoresist mold structure.

64. The method of claim 58 wherein the first substrate is a silicon wafer and the steps of forming the mold further comprise:
depositing photoresist onto the surface of the silicon wafer substrate;
patterning the photoresist using a mask that defines the chambers and capture sites; and
developing the photoresist mold structure.

65. A method for manipulating a cell population, the method comprising:
providing a cell manipulation apparatus with an array of sites across at least one substrate in a pattern, each site configured to hold one cell, and each site including a capture mechanism capable of capturing one cell and a release mechanism comprising at least one microbubble actuator for selectively releasing the cell from the site,
introducing a fluid medium containing a plurality of cells onto the apparatus,
capturing a cell in at least one well,
assessing at least one property of the captured cell, and
selectively releasing the captured cell based on the assessment.

66. The method of claim 65, wherein the step of assessing a property further comprises introducing at least one fluid reagent across the captured cell.

67. The method of claim 66, wherein the step of introducing at least one fluid reagent further comprises selectively introducing a fluid across the captured cell.

68. The method of claim 66, wherein the step of introducing at least one fluid reagent further comprises introducing a fluid gradient across the captured cell.

69. The method of claim 66, wherein the step of introducing at least one fluid reagent further comprises introducing a plurality of fluid reagents across the captured cell.
